(19) United States
(12) Patent Application Publication
(10) Pub. No.: US 2017/0056304 A1
(43) Pub. Date: Mar. 2, 2017

(54) PARTICULATE HYDROXYAPATITE COMPOSITIONS AND METHODS FOR PREPARING THE SAME

(71) Applicant: Honeywell International Inc., Morris Plains, NJ (US)

(72) Inventors: Roland Friedemann, Seelze (DE); Christian Werner, Hannover (DE)

(21) Appl. No.: 15/237,810

(22) Filed: Aug. 16, 2016

Related U.S. Application Data
(60) Provisional application No. 62/210,259, filed on Aug. 26, 2015.

(51) Int. Cl. A61K 8/24; A61K 8/34; A61K 9/16; A61K 33/42; A61K 47/10; A61K 9/06; A61Q 11/00; A61K 8/02

(52) U.S. Cl. CPC A61K 8/24; A61Q 11/00; A61K 8/34; A61K 8/0216; A61K 8/0241; A61K 33/42; A61K 47/10; A61K 9/06; A61K 9/16; A61K 2800/412

(57) ABSTRACT

A method for preparing a fast-drying particulate hydroxyapatite composition includes the steps of reacting a calcium salt aqueous solution with a phosphate salt aqueous solution to obtain a product solution including hydroxyapatite particles in aqueous suspension and a dissolved byproduct salt; purifying the product solution by removing at least a portion of the dissolved byproduct salt to form a purified product; and dehydrating the purified product by diluting the purified product with a non-water fluid to form a diluted product in a water/non-water mixed solution and subsequently removing at least a portion of the water/non-water mixed solution to form a dehydrated product.

### REFERENCE TO RELATED APPLICATION

This application claims benefit of provisional patent application 62/210,259 filed Aug. 26, 2015, which is incorporated herein by reference in its entirety.

### TECHNICAL FIELD

The present disclosure generally relates to hydroxyapatite compositions and methods for preparing the same. More particularly, the present disclosure relates to fast-drying particulate hydroxyapatite compositions and methods for preparing the same.

### BACKGROUND

Hydroxyapatite is a naturally occurring mineral form of calcium apatite with the formula Ca₅(PO₄)₃(OH). Hydroxyapatite is the main mineral of which dental enamel and dentin are composed. Accordingly, hydroxyapatite finds commercial application in medical and cosmetic preparations that are intended to be applied to the teeth.

Hydroxyapatite can be synthesized via several methods such as wet chemical deposition, biomimetic deposition, sol-gel route (wet-chemical precipitation), or electrodeposition. For medical and cosmetic preparations, it is desirable that the synthesized hydroxyapatite particles be small enough to enter the micro-pores of the teeth to allow the particles to achieve their desired medical/cosmetic effect. To achieve a small particulate size, the prior art teaches that hydroxyapatite synthesis via the wet-chemical route can be improved by power ultrasound. The ultrasonically-assisted synthesis (sono-synthesis) of hydroxyapatite is a technique to produce nanostructured hydroxyapatite, such as nano-crystalline hydroxyapatite, the particles of which are sufficiently small to enter the micro-pores of the teeth.

A remaining problem in the art, however, is that the ultrasonically-assisted wet-chemical hydroxyapatite preparations are water-based. In some applications, water is an undesirable medium for application of hydroxyapatite to the teeth because water-based preparations dry too slowly. For example, if a hydroxyapatite preparation is intended for use as a brightening varnish to the teeth, it is desirable for the preparation to dry as quickly as possible such that the hydroxyapatite particles adhere faster and more strongly to the teeth. In another example, if a hydroxyapatite preparation is intended for use in teeth re-mineralization, it is again desirable for the preparation to dry as quickly as possible such that the particles remain well-adhered within the micro-pores of the teeth. To date, the prior art is deficient of any suitable fine-particulate hydroxyapatite preparations that are not water-based.

Accordingly, it would be desirable to provide hydroxyapatite preparations, and methods for preparing the same, which are not water based, and are relatively faster-drying than water-based compositions. Moreover, it would be desirable to provide such preparations with a sufficiently small particle size of the hydroxyapatite to allow for use in medical and cosmetic applications, such as application to the teeth. Other desirable features and characteristics of this disclosure will become apparent from the subsequent detailed description and the appended claims, taken in conjunction with this background of the inventive subject matter.

### BRIEF SUMMARY

Generally disclosed are fast-drying particulate hydroxyapatite compositions and methods for preparing the same. In one exemplary embodiment, a method for preparing a fast-drying particulate hydroxyapatite composition includes the steps of reacting a calcium salt aqueous solution with a phosphate salt aqueous solution to obtain a product solution including hydroxyapatite particles in aqueous suspension and a dissolved byproduct salt; purifying the product solution by removing at least a portion of the dissolved byproduct salt to form a purified product; and dehydrating the purified product by diluting the purified product with a non-water fluid to form a diluted product in a water/non-water mixed solution and subsequently removing at least a portion of the water/non-water mixed solution to form a dehydrated product

In another exemplary embodiment, a fast-drying medical or cosmetic paste composition of hydroxyapatite particles includes about 10 wt.-% to about 30 wt.-% of the hydroxyapatite particles, the hydroxyapatite particles having a D₅₀ mean particle size of about 5.0 microns to about 8.0 microns; about 20 wt.-% or less of water; about 1000 wt.-ppm of byproduct impurity salt; and a balance of non-water fluid.

In yet another exemplary embodiment, a method for preparing a fast-drying particulate hydroxyapatite composition includes the steps of reacting a calcium chloride salt aqueous solution with a potassium phosphate salt aqueous solution to obtain a product solution including hydroxyapatite particles in aqueous suspension and a dissolved potassium chloride byproduct salt, wherein the hydroxyapatite particles have a D₅₀ mean particle size of about 0.5 micron to about 5.0 microns; purifying the product solution by removing at least a portion of the dissolved byproduct salt to form a purified product, wherein purifying includes performing at least twice each the steps of centrifuging, decanting, and washing the product solution; and dehydrating the purified product by diluting the purified product with a non-water fluid including ethanol to form a diluted product in a water/non-water mixed solution and subsequently removing at least a portion of the water/non-water mixed solution to form a dehydrated product, wherein dehydrating includes reducing a weight percent of water in the purified product to less than about 20%, and wherein dehydrating to form the dehydrated product includes forming a product with hydroxyapatite particles in a paste having a D₅₀ mean particle size of about 5.0 microns to about 8.0 microns.

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

### DETAILED DESCRIPTION

The following detailed description is merely exemplary in nature and is not intended to limit the inventive subject matter or the application and uses of the inventive subject matter. Furthermore, there is no intention to be bound by any theory presented in the preceding background or the following detailed description.

The present disclosure provides non-water-based hydroxyapatite compositions and methods for preparing the same that have desirably small particle sizes for use in medical and cosmetic applications. The compositions dry relatively more quickly than water-based compositions, and thus are particularly suitable for application to the teeth as cosmetic teeth brightening products and as teeth restorative/regenerative products. The compositions are prepared by first reacting a calcium salt with a phosphate salt in aqueous solution to form a hydroxyapatite suspension in water. The hydroxyapatite suspension in water is then purified to remove reaction byproducts. The purified product is then dehydrated by diluting the purified product with a non-water-based fluid, and then re-concentrating the product by removing some of the resulting water/non-water fluid mixture to achieve a relatively lesser concentration of water. This procedure desirably allows the hydroxyapatite particles to remain sufficiently small throughout the reaction, purification, and dehydration stages so that the need for subsequent processing to reduce particle size is substantially lessened or entirely eliminated.

The synthesis of a non-water-based particulate hydroxyapatite preparation includes, according to some embodiments, the provision of a calcium salt and a phosphate salt as reactants. Non-limiting examples of suitable calcium salts include calcium chloride, calcium phosphate, calcium nitrate, calcium ethylenediaminetetraacetate, calcium carbonate, calcium acetate, calcium bromide, calcium fluoride, calcium iodide, calcium citrate, calcium hydroxide, calcium oxylate, calcium sulfate, calcium ethoxide, or a combination thereof. In some embodiments, calcium chloride is chosen as the calcium salt. Non-limiting examples of suitable phosphate salts include potassium phosphate, calcium phosphate, sodium phosphate, ammonium phosphate, lithium phosphate, magnesium phosphate, triethyl phosphate, or a combination thereof. In some embodiments, potassium phosphate is chosen as the phosphate salt. Either or both of the calcium salt and the phosphate salt may be provided in the form stated above, or may be synthesized according to well-known methods.

The calcium salt and the phosphate salt may thereafter be provided in an aqueous solution. For example, the calcium salt may be dissolved in a first solution, and the phosphate salt may be dissolved in a second solution. The salts may be maintained in separate solutions to prevent premature reaction of the salts outside of the controlled reaction conditions as will be described in greater detail below. Solubility of the salts may be appropriately controlled by the ratio of water to salt in the aqueous solution, the pH of the aqueous solution, the temperature of the aqueous solution, and agitation of aqueous solutions (for example by stirring), among other factors as known in the art. In exemplary embodiments, the concentration of each salt solution is independently maintained from about 0.5 molar to about 5.0 molar, the pH is independently maintained from about 4.0 to about 12.0, the temperature is independently maintained from about 15° C. to about 100° C., and agitation may or may not be applied.

In some embodiments, synthesis of the hydroxyapatite preparation continues with contacting (reacting) the calcium salt solution with the phosphate salt solution to form the hydroxyapatite particles. Various methods of contacting/reacting the salt solutions with one another may be employed. For example, in one embodiment, a micro mixer is employed for this purpose. As is known in the art, a micro mixer receives two or three fluid streams and brings them into intimate contact at a controlled, micro-scale level for highly-accurate mixing. In this example, thus, the micro mixer receives as its first fluid stream the calcium salt solution and as its second fluid stream the phosphate salt solution. The micro mixer efficiently mixes the two solutions together, reacting the calcium salt with the phosphate salt to form the hydroxyapatite particles, which are expelled from the mixer as part of a product fluid stream, the product fluid stream also containing the complementary ions of the calcium salt and the phosphate salt. In the exemplary use of calcium chloride and potassium phosphate, the product fluid stream from the micro mixer will thus also include potassium chloride in solution, in addition to the hydroxyapatite particles. The product fluid stream may be agitated, for example by stirring, for a period of time after the reaction to hydroxyapatite is complete. This period of time may be from about 5 minutes to about 5 hours, as desired. Upon completion of the foregoing procedures, the product solution may be at a pH of from about 8.0 to about 11.0, and have suspended hydroxyapatite particles with a D₅₀ mean particle size of from about 0.5 μm to about 5.0 μm, such as from about 1.0 μm to about 3.0 μm. This mean particle size is generally recognized as suitable for use in medical and cosmetic applications, such as teeth brightening and/or regeneration. The amount of hydroxyapatite in solution, depending on the initial concentration of the salt solutions, generally is from about 0.5% by weight to about 5.0 percent by weight, such as from about 1.0% by weight to about 3.0% by weight.

In another embodiment, subsequent to or as part of the mixing process, ultrasonic vibrations may be applied to the solution(s). As is known in the art, ultrasonic vibrations typically refer to those with a frequency of about 20 kHz or greater. Application of the ultrasonic vibrations during or subsequent to the mixing process may be performed for a time period of from about 10 minutes to about 100 minutes, for example. Furthermore, the power used to generate the ultrasonic vibrations may be from about 10 Watts to about 500 Watts.

As noted above, the product solution includes dissolved by-product salts in solution, such as potassium chloride, which may be considered impurities. Thus, in some embodiments of the present disclosure, the preparation of the hydroxyapatite composition continues with purification procedures. In some embodiments, a manual centrifuge may be employed for this purpose. The manual centrifuge is operated to collect the hydroxyapatite suspension at the bottom of the containment device, and thereafter the aqueous solution containing the by-product salt (but not the hydroxyapatite) is decanted. Thereafter, the hydroxyapatite particles that remain in the containment device after decanting may be washed with distilled or otherwise purified water. The centrifuge/decant/washing process may thereafter be repeated one, two, three, or more times to achieve a desired level of purity of the hydroxyapatite product. In another embodiment, a continuous centrifuge may be employed for the purpose of purifying the hydroxyapatite product. Using a continuous centrifuge, as is known in the art, the above-described separate steps of centrifuging, decanting, and washing, are performed essentially simultaneously and continuously, which makes this method more suitable for commercial-scale applications. The continuous centrifuge process is performed for a period of time sufficient to achieve the desired level of purity of the hydroxyapatite product. As a result of the purification process (according to either embodiment), the resulting hydroxyapatite product typically is a paste with from about 20% to about 40%, such as from about 25% to about 30%, of the composition by weight being hydroxyapatite particles, and the substantial balance being water, with less than about 2000 ppm, such as less than about 1000 ppm, of the impurity salt species (such as chloride) remaining.

Subsequent to the aforementioned contacting/reacting and purification procedures, as noted above, the hydroxyapatite is present as a water-based paste. As previously noted, in some applications, water-based preparations of hydroxyapatite are not desirable due to their relatively slow drying upon application to the teeth. Accordingly, further procedures of the presently-described method, as set forth below, convert the obtained aqueous suspension of hydroxyapatite to a non-water-based preparation. As used herein, the term “non-water-based preparation” refers to a hydroxyapatite preparation wherein a fluid medium is employed, at least partially in place of water, which dries relatively faster than water. From a physical standpoint, this means that the fluid medium has a relatively higher vapor pressure and/or a relatively lower boiling point as compared to water (all other variables assumed constant). Further, in some embodiments, where application to the teeth or other medical/cosmetic purposes are intended, the fluid medium desirably is a pharmaceutically and/or cosmetically-acceptable fluid. Examples of such fluids include, but are not limited to, various volatile oils and alcohols. In one particular embodiment, the fluid medium is ethanol.

As further used herein, reference to the fluid medium being used at least partially in place of water means that the maximum allowable amount of water that may remain in the hydroxyapatite preparations of the present disclosure varies depending on the particular application, and on the desired speed of drying for such application. In some embodiments, water is limited to less than about 50%, by weight, of the overall composition. In other embodiments, water is limited to less than about 20% or less than about 10%. In more demanding applications where fast-drying is highly-desired, water is limited to less than about 5%, less than about 2%, or less than about 1% of the overall composition by weight.

Accordingly in some embodiments, the preparation process of the present disclosure thus continues with procedures to at least partially replace the water in the above-described hydroxyapatite paste, in what is referred to herein as dehydrating procedures. In one embodiment, the dehydrating procedures may be accomplished by adding to the paste the non-water fluid, such as ethanol, in an amount sufficient to dilute the concentration of hydroxyapatite in the composition to from about 1% to about 10%, such as to about 5%, by weight. The fluid part of this composition may be referred to as a water/non-water mixed solution. Thereafter, similar centrifuge/decanting procedures, as described above with regard to the purification procedures, may be used to re-obtain a paste with a hydroxyapatite concentration of about from 10% to about 30%, such as about from 15% to about 25%. For example, either of the manual or continuous centrifuging procedures as described above may be employed for this purpose. Accordingly, after decanting, the amount of water in the paste is reduced by about from 50% to about 80%, depending on the amount of the non-water fluid that is added (i.e., the dilution of the hydroxyapatite composition). This procedure of dilution with the non-water fluid and subsequent centrifuging/decanting may be performed one, two, three, or more times, depending on the maximum acceptable amount of water in the final preparation, as described above, which may be from less than about 50% to less than about 1%, depending on the application. For example, if the procedure of dilution with the non-water fluid and subsequent centrifuging/decanting is performed two times, it may be expected that the resultant hydroxyapatite paste includes from about 15% to about 25% hydroxyapatite, from about 3% to about 6% water, and a balance of non-water fluid (e.g., ethanol), all by weight, with by-product salt concentrations being less than about 1000 ppm.

Accordingly, the above-described method for preparing a fast-drying particulate hydroxyapatite composition may be characterized as including the steps of reacting a calcium salt aqueous solution with a phosphate salt aqueous solution to obtain a product solution including hydroxyapatite particles in aqueous suspension and a dissolved byproduct salt; purifying the product solution by removing at least a portion of the dissolved byproduct salt to form a purified product; and dehydrating the purified product by diluting the purified product with a non-water fluid to form a diluted product in a water/non-water mixed solution and subsequently removing at least a portion of the water/non-water mixed solution to form a dehydrated product. Further, the resulting purified, dehydrated hydroxyapatite paste composition may be characterized as having from about 10 wt.-% to about 30 wt.-% of the hydroxyapatite particles, the hydroxyapatite particles having a D50 mean particle size of from about 5.0 microns to about 8.0 microns, about 20 wt.-% or less of water, about 1000 wt.-ppm of byproduct impurity salt, and a balance of non-water fluid, for example ethanol.

It should be noted that the above-described processes allow the hydroxyapatite particles to remain desirably small. For example, it has been discovered that if the hydroxyapatite is allowed to dry, such that the water content drops below about 25% by weight, much larger crystal structures re-form, and the subsequent processes required (upon re-dilution) to reduce the particle size are both time, energy, and cost intensive. Thus, the processes described in this disclosure beneficially allow the hydroxyapatite particles to be formed initially at the appropriate size in water, and thereafter remain the appropriate size through the transformation from a water-based composition to a non-water-based composition. For example, upon final preparation of the non-water based hydroxyapatite paste composition as described above, the hydroxyapatite particle size may be expected to be from about 5.0 μm to about 8.0 μm, which is sufficiently small for most dental applications. Of course, further ultrasonic procedures may thereafter be employed if a further reduced particle size is desired, however it should be appreciate that the amount of time needed to do so would be significantly reduced as compared to if larger hydroxyapatite crystals had been allowed to re-form.

The resulting non-water-based hydroxyapatite paste composition may be used in the preparation of a variety of medical and cosmetic compositions for application directly to the teeth, such as teeth brightening pastes and teeth restorative/regenerative pastes. Other suitable uses will be appreciated by those having ordinary skill in the art.

### ILLUSTRATIVE EXAMPLE

The present disclosure is now illustrated by the following non-limiting example. It should be noted that various changes and modifications can be applied to the following example and processes without departing from the scope of this disclosure, which is defined in the appended claims. Therefore, it should be noted that the following example should be interpreted as illustrative only and not limiting in any sense.

A hydroxyapatite in ethanol paste was prepared according to the following exemplary procedures:

### Preparation of Reactants

Potassium phosphate (K₃PO₄), as a reactant, was prepared by the reaction of phosphoric acid and potassium hydroxide. A 5 L glass beaker was filled with about 3 L of deionized water. Under stirring, first 138.0 g of phosphoric acid as an 85% aqueous solution and second 448.0 g of potassium hydroxide as a 45% aqueous solution were added to the 5 L glass beaker. After that the beaker was filled to the 5 L level with additional water. The result was 5 L of potassium phosphate in aqueous solution.

Calcium chloride (CaCl₂), as a reactant, was prepared by filling a 5 L glass beaker with about 3 L of deionized water, and stirring in 292.6 g of CaCl₂.2H₂O. After that the beaker was filled to the 5 L level with additional water. The result was 5 L of calcium chloride in aqueous solution.

### Reaction Apparatus and Setup

Micro mixer, tube pump, 3×PVC-tubes, Y-Fitting, 10 L PE-Canister with stirrer. In both 5 L beakers (i.e., those containing the K₃PO₄ and the CaCl₂) one PVC-tube was submerged. The tubes were connected by the Y-fitting with the micro mixer. After the micro mixer the third PVC tube was connected and lead via the tube pump to the canister.

### Reaction Procedure

The reaction proceeded according to the following stoichiometric chemical formula:

5 CaCl₂+3 K₃PO₄+H₂O→Ca₅(PO₄)₃OH+9 KCl+HCl

The tube pump was set to maximum capacity (i.e., 400 ml/min) and started. Both solutions were sucked in the tubes and were mixed in the micro mixer. Direct after the contact of the two solutions the reaction took place and a white suspension of hydroxyapatite was formed. The suspension was stirred for an additional hour.

Result of Reaction: 10 L hydroxyapatite suspension 2 wt.-% in water; pH value: 9-10; particle size: D₅₀ (after 4 min ultrasonic processing) 1-3 μm.

### Purification

For purification the solid hydroxyapatite settled for about an hour and the remaining water (KCl solution) was decanted. Two procedures were conducted:

Manual Centrifuge: The suspension was filled into centrifuge beakers, centrifuged, washed/stirred with water, and centrifuged again. Washing and centrifugation was repeated several times until the chloride value was as desired. The product was a paste with about 18-25 wt.-% hydroxyapatite. Continuous Centrifuge: A lab decanter from LEMITEC® was used. The 2 wt.-% solution was concentrated to 25-30 wt.-% hydroxyapatite in one step. To remove the byproducts the paste was washed/stirred with water to an approximately 5 wt.-% suspension, and centrifuged again. This purification process was repeated 2 times to obtain a paste with a chloride assay of less than 1000 ppm by weight.

Result of Purification: 600 g hydroxyapatite paste with 25-30 wt.-% hydroxyapatite, assay Cl: <1000 wt.-ppm.

### Dehydration

The water was removed and replaced by ethanol (ABSOLUT PURANAL®). Therefore the hydroxyapatite paste was diluted in ethanol to a concentration of approximately 5 wt.-% hydroxyapatite in liquid. This suspension was centrifuged to obtain a paste with 15-25 wt.-% hydroxyapatite in ethanol with remaining water. It was suspended again in ethanol to a concentration of approximately 5 wt.-% hydroxyapatite in liquid and centrifuged again to obtain a white paste.

Result of Dehydration: 500 g hydroxyapatite in Ethanol; assay H₂O: 4.4 wt.-%; assay Cl: <1000 wt.-ppm; particle size distribution D₅₀ after 0 min. ultrasonic processing: 7.7 μm, after 1 min.: 7.2 μm, after 2 min.: 6.7 μm, after 4 min.: 5.4 μm.

Accordingly, the present disclosure has provided non-water-based hydroxyapatite compositions and methods for preparing the same that have desirably small particle sizes for use in medical and cosmetic applications. The compositions dry relatively more quickly than water-based compositions, and thus are particularly suitable for application to the teeth as cosmetic teeth brightening products and as teeth restorative/regenerative products.

While at least one exemplary embodiment has been presented in the foregoing detailed description of the inventive subject matter, it should be appreciated that a vast number of variations exist. It should also be appreciated that the exemplary embodiment or exemplary embodiments are only examples, and are not intended to limit the scope, applicability, or configuration of the inventive subject matter in any way. Rather, the foregoing detailed description will provide those skilled in the art with a convenient road map for implementing an exemplary embodiment of the inventive subject matter. It being understood that various changes may be made in the function and arrangement of elements described in an exemplary embodiment without departing from the scope of the inventive subject matter as set forth in the appended claims.

What is claimed is:

1. A method for preparing a fast-drying particulate hydroxyapatite composition comprising the steps of:
reacting a calcium salt aqueous solution with a phosphate salt aqueous solution to obtain a product solution comprising hydroxyapatite particles in aqueous suspension and a dissolved byproduct salt;
purifying the product solution by removing at least a portion of the dissolved byproduct salt to form a purified product; and
dehydrating the purified product by diluting the purified product with a non-water fluid to form a diluted product in a water/non-water mixed solution and subsequently removing at least a portion of the water/non-water mixed solution to form a dehydrated product.

2. The method of claim 1, wherein reacting the calcium salt aqueous solution comprises reacting an aqueous solution of calcium chloride with the phosphate salt aqueous solution.

3. The method of claim 1, wherein reacting the calcium salt aqueous solution with the phosphate salt aqueous solution comprises reacting the calcium salt aqueous solution with an aqueous solution of potassium phosphate.

4. The method of claim 1, wherein reacting to obtain the product solution comprises reacting to obtain a solution comprising a dissolved potassium chloride byproduct salt.

5. The method of claim 1, wherein reacting to obtain the product solution comprises reacting to obtain a solution comprising hydroxyapatite particles in suspension having a D₅₀ mean particle size of about 0.5 micron to about 5.0 microns.

6. The method of claim 1, wherein purifying comprises centrifuging, decanting, and washing the product solution.

7. The method of claim 6, wherein the step of purifying is performed two or more times.

8. The method of claim 1, wherein dehydrating the purified product comprises diluting the purified product to a hydroxyapatite composition by weight of from about 1% to about 10%.

9. The method of claim 1, wherein diluting with the non-water fluid comprises diluting with a fluid having a vapor pressure greater than a vapor pressure of water or having a boiling point less than a boiling point of water.

10. The method of claim 9, wherein diluting with the non-water fluid comprises diluting with a pharmaceutically or cosmetically acceptable fluid.

11. The method of claim 10, wherein diluting with the non-water fluid comprises diluting with a volatile oil or an alcohol.

12. The method of claim 11, wherein diluting with the non-water fluid comprises diluting with ethanol.

13. The method of claim 1, wherein dehydrating comprises reducing a weight percent of water in the purified product to less than about 20%.

14. The method of claim 13, wherein dehydrating comprises reducing the weight percent of water in the purified product to less than about 5%.

15. The method of claim 1, wherein dehydrating to form the dehydrated product comprises forming a product with hydroxyapatite particles in a paste having a D₅₀ mean particle size of from about 5.0 microns to about 8.0 microns.

16. A fast-drying medical or cosmetic paste composition of hydroxyapatite particles comprising:
from about 10 wt.-% to about 30 wt.-% of the hydroxyapatite particles, the hydroxyapatite particles having a D₅₀ mean particle size of from about 5.0 microns to about 8.0 microns;
about 20 wt.-% or less of water;
about 1000 wt.-ppm of byproduct impurity salt; and
a balance of non-water fluid.

17. The composition of claim 16, wherein the hydroxyapatite particles are present in an amount of from about 15 wt.-% to about 25 wt.-%.

18. The composition of claim 16, wherein the water is present in an amount of about 5 wt.-% or less.

19. The composition of claim 16, wherein the non-water fluid is a cosmetically or pharmaceutically acceptable fluid that has a vapor pressure greater than a vapor pressure of water or a boiling point less than a boiling point of water.

20. A method for preparing a fast-drying particulate hydroxyapatite composition comprising the steps of:
reacting a calcium chloride salt aqueous solution with a potassium phosphate salt aqueous solution to obtain a product solution comprising hydroxyapatite particles in aqueous suspension and a dissolved potassium chloride byproduct salt, wherein the hydroxyapatite particles have a D₅₀ mean particle size of about 0.5 micron to about 5.0 microns;
purifying the product solution by removing at least a portion of the dissolved byproduct salt to form a purified product, wherein purifying comprises performing at least twice each the steps of centrifuging, decanting, and washing the product solution; and
dehydrating the purified product by diluting the purified product with a non-water fluid comprising ethanol to form a diluted product in a water/non-water mixed solution and subsequently removing at least a portion of the water/non-water mixed solution to form a dehydrated product, wherein dehydrating comprises reducing a weight percent of water in the purified product to less than about 20%, and wherein dehydrating to form the dehydrated product comprises forming a product with hydroxyapatite particles in a paste having a D₅₀ mean particle size of about 5.0 microns to about 8.0 microns.
